(19) United States
(12) Patent Application Publication
(10) Pub. No.: US 2026/0200972 A1
(43) Pub. Date: Jul. 16, 2026

(54) PERFUSION AND METABOLIC RESTORATION OF ORGANS

(71) Applicant: Bexorg, Inc., New Haven, CT (US)

(72) Inventor: Zvonimir Vrselja, New Haven, CT (US)

(21) Appl. No.: 19/449,063

(22) Filed: Jan. 14, 2026

Related U.S. Application Data
(60) Provisional application No. 63/745,574, filed on Jan. 15, 2025.

(51) Int. Cl. C07K 1/34; B01D 21/26; B01D 61/16; B01D 61/18; B01D 61/22; B01D 61/58; B01D 69/02; C07K 14/435

(52) U.S. Cl. CPC C07K 1/34; B01D 21/262; B01D 61/16; B01D 61/18; B01D 61/22; B01D 61/58; B01D 69/02; C07K 14/43536; B01D 2311/04; B01D 2313/18; B01D 2313/221; B01D 2313/243; B01D 2313/903; B01D 2315/10; B01D 2315/16; B01D 2325/20

(57) ABSTRACT

Systems, devices, and methods for managing air gaps in a semiconductor device are provided. In one aspect, a method includes removing coelomic fluid from an exterior surface of the worms using a drying agent; removing blood from the worms; performing centrifugation on the blood to generate a blood supernatant; concentrating, by a tangential flow filtration system with a single membrane, the blood supernatant to form a concentrated blood product; and performing, by the tangential flow filtration system, tangential flow filtration of a diafiltration mixture comprising the concentrated blood product.

### TECHNICAL FIELD

This specification relates to methods, systems, and compositions for manufacturing a cell free oxygen carrier composition.

### BACKGROUND

Perfusion in mammalian brains refers to the process of delivering perfusate (e.g., blood or an artificial perfusate) to the brain tissue to supply inputs such as oxygen, glucose, and nutrients, while removing outputs such as carbon dioxide and metabolic wastes.

### SUMMARY

In one aspect of the disclosure, a method of isolating extracellular hemoglobin protein from worms includes: removing coelomic fluid from an exterior surface of the worms using a drying agent; removing blood from the worms; performing centrifugation on the blood to generate a blood supernatant; and concentrating the blood supernatant to form a concentrated blood product and performing tangential flow filtration of a diafiltration solution including the concentrated blood product by a tangential flow filtration system with a single membrane.

Implementations can include one or more of the following features in any combination.

In some implementations, the drying agent includes cornstarch, and removing the coelomic fluid from the exterior surface of the worms includes coating an outer surface of the worms with the cornstarch.

In some implementations, removing the coelomic fluid from the exterior surface of the worms includes rinsing the cornstarch from the outer surface of the worms.

In some implementations, removing the blood from the worms includes grinding the worms by a grinder to obtain ground worms and draining the blood from the ground worms.

In some implementations, draining the blood from the ground worms includes placing the ground worms in a filter bag and flowing the blood through the filter bag into a receptacle.

In some implementations, the method includes before concentrating the blood supernatant by the tangential flow filtration system, filtering the blood supernatant.

In some implementations, filtering the blood supernatant comprises flowing the blood supernatant through a 0.22 µm filter.

In some implementations, the method includes before concentrating the blood supernatant by the tangential flow filtration system, diluting the blood supernatant with a buffer agent and storing the diluted blood supernatant at a low temperature.

In some implementations, concentrating the blood supernatant to form the concentrated blood product includes: flowing the blood supernatant from a feed receptacle through the single membrane to generate a concentrated blood solution, where the single membrane includes a 750 kDa one-way filter; and flowing the concentrated blood solution back to the feed receptacle.

In some implementations, the method includes adding a buffer solution to the concentrated blood product to obtain the diafiltration mixture.

In some implementations, performing the diafiltration on the diafiltration mixture includes performing continuous diafiltration on the diafiltration mixture.

In some implementations, the buffer solution is configured to be automatically added to a feed receptacle of the tangential flow filtration system throughout the continuous diafiltration on the diafiltration mixture to maintain a constant volume of the diafiltration mixture within the tangential flow filtration system.

In some implementations, the buffer solution is configured to be automatically added to a feed receptacle of the tangential flow filtration system based at least partly on one or more signals received from at least one scale of the tangential flow filtration system.

In some implementations, the single membrane is a 750 kDa one-way filter.

In some implementations, performing the diafiltration on the diafiltration mixture includes controlling a temperature of the diafiltration mixture throughout the diafiltration based on one or more signals received from a temperature sensor.

In some implementations, controlling the temperature of the diafiltration mixture includes maintaining the temperature of the diafiltration mixture at 10℃.

In some implementations, performing the diafiltration on the diafiltration mixture includes controlling a flow rate of the diafiltration mixture based on one or more signals received from a flow sensor.

In some implementations, controlling the flow rate of the diafiltration mixture includes maintaining the flow rate of the diafiltration mixture at about 300mL/min.

In some implementations, performing the diafiltration on the diafiltration mixture includes controlling a pressure of the diafiltration mixture passing through the single membrane based on one or more signals received from at least one pressure sensor.

In some implementations, controlling the pressure of the diafiltration mixture passing through the single membrane includes controlling a pinch valve of the tangential flow filtration system.

In some implementations, controlling the pressure of the diafiltration mixture includes maintaining the pressure at about 200 mmHg.

In some implementations, the extracellular hemoglobin protein is erythrocruorin.

In some implementations, the worms include Lumbricus terrestris earthworms.

In another aspect, a system for isolating extracellular hemoglobin protein from worms, the system including: a device for removing blood from the worms; a centrifuge configured to perform centrifugation on the blood to generate a blood supernatant; and a tangential flow filtration system with a single membrane. The tangential flow filtration system is configured to concentrate the blood supernatant to form a concentrated blood product and to perform diafiltration on a diafiltration mixture including the concentrated blood product to isolate the extracellular hemoglobin protein from the blood.

Implementations can include one or more of the following features in any combination.

In some implementations, the device for removing blood from the worms includes a meat grinder.

In some implementations, the single membrane is a 750 kDa one-way filter.

In some implementations, the tangential flow filtration system includes: a feed receptacle; a fluid line fluidly coupling the feed receptacle to the single membrane; a peristaltic pump configured to flow the diafiltration mixture from the feed receptacle, along the fluid line, and through the single membrane; and at least one flow sensor fluidly coupled to the fluid line downstream of the single membrane, where the peristaltic pump is configured to be controlled based at least in part on one or more signals generated by the at least one flow sensor.

In some implementations, the tangential flow filtration system includes: a feed receptacle; a fluid line fluidly coupling the feed receptacle to the single membrane; a pinch valve coupled to the fluid line downstream of the single membrane; and at least one pressure sensor fluidly coupled to the fluid line, where the pinch valve is configured to be controlled based at least in part on one or more signals generated by the at least one pressure sensor.

In some implementations, the at least one pressure sensor includes a first pressure sensor coupled to the fluid line upstream of the single membrane and a second pressure sensor coupled to the fluid line downstream of the single membrane.

In some implementations, the tangential flow filtration system includes: at least one temperature sensor; and a heat exchanger system, where the heat exchanger system is configured to control a temperature of the diafiltration mixture based on one or more signals received from the at least one temperature sensor.

In some implementations, the at least one temperature sensor includes a first temperature sensor coupled to a fluid line upstream of the single membrane and a second temperature sensor coupled to the fluid line downstream of the single membrane.

In some implementations, the extracellular hemoglobin protein is erythrocruorin.

In some implementations, the worms include Lumbricus terrestris earthworms.

Implementations can include one or more of the following advantages. For example, some of the systems and methods of extracting extracellular hemoglobin protein erythrocruorin (also called LtEc in the present disclosure) from Lumbricus terrestris (commonly known as earthworm) that are described herein can reduce potential protein denaturization and aggregation, thereby improving the extraction yield. In some implementations, the extracted LtEc is pure (e.g., without non-specific bands by visual inspection of gel electrophoresis), sterile, and stable for long term storage. In some cases, systems and methods described in this disclosure can produce at least 40 percent (e.g., 40-50 percent) greater yield of pure LtEc compared to other known systems and methods. In certain cases, for example, the yield can be increased from about 4.8g to about 6.8g of pure LtEc for every 500 earthworms.

In addition, certain systems and methods described herein utilize cornstarch to remove mucus from earthworms before obtaining crude blood from the earthworms. The mucus can be one of the factors that cause protein aggregation during the LtEc extraction process. Without wishing to be bound by theory, it is believed that the removal of the mucus by cornstarch can sequester coelomic enzymes in the mucus that contribute to LtEc aggregation. LtEc aggregation may clog a tangential flow filtration (TFF) system when they are directed to pass through a filter membrane of the TFF system, leading to low extraction yield of LtEc. Therefore, removing mucus can help reduce protein aggregation and improve the extraction yield.

Moreover, in some implementations, a grinder is employed to cut earthworms into pieces. Compared to crushing the earthworms by a blender to create a homogenous puree, slicing the earthworms with the grinder allows the crude blood of the earthworms to drain with less interaction between LtEc and other mucosal proteins, thereby reducing protein aggregation and further improving yields. In addition, the grinder may help reduce the formation of air bubbles in the blood puree. Its use can also enable scalable production of crude blood. In certain implementations, for example, more than 500 worms can be processed simultaneously using the grinder.

Additionally, crude blood obtained from earthworms can be purified through centrifugation for about 20 mins. Compared to 1.5 hours used in other extraction methods, this reduced centrifugation time can help decrease the potential aggregation between LtEc and other worm proteins. Further, a diluted blood supernatant can be stored in bottles at a low temperature between 0 ℃ and 8℃ (e.g., 4℃) for extended time periods (e.g., up to four hours) to limit protein denaturation. This also facilitates the preparation of larger solution batches for the TFF processing in subsequent stages.

In some implementations, a continuous diafiltration process using the TFF system for LtEc extraction is automated. Automating the diafiltration process with a computer system can significantly reduce labor time requirement. In certain cases, the automated process requires only about one-fourth of the time compared to other extraction methods. This improved efficiency can lower labor and production costs when scaled up.

In some implementations, perfusate solution utilizing LtEc extracted and purified using the systems and methods described herein can enable perfusions of organs (e.g., human brains) that last two weeks or longer.

The details of certain implementations are set forth in the accompanying drawings and the description below. Other aspects, features, and advantages will be apparent from the description and drawings, and from the claims.

### BRIEF DESCRIPTION OF DRAWINGS

FIG. 1 is a schematic illustration of a process that includes (i) manufacturing a perfusate including erythrocruorin (LtEc) that has been isolated from Lumbricus terrestris (commonly known as earthworms), and then (ii) using the perfusate to perfuse a human brain.

FIG. 2. is flow diagram of a process of manufacturing the LtEc of FIG. 1.

FIGS. 3A through 3E depict an example process of removing coelomic fluid from an exterior surface of earthworms using cornstarch for use in the LtEc manufacturing process depicted in FIG. 2.

FIG. 4 depicts an example process of removing blood from earthworms for use in the LtEc manufacturing process depicted in FIG. 2.

FIG. 5 is a schematic illustration of a tangential flow filtration system for use in the LtEc manufacturing process depicted in FIG. 2.

FIG. 6 is a perspective view of the tangential flow filtration system of FIG. 5.

FIG. 7 depicts an example gel characterization of the LtEc generated using the process depicted in FIG. 2.

FIG. 8 is a block diagram of an example computer system by which a computer system of the tangential flow filtration system of FIG. 5 can be implemented.

FIG. 9 is a schematic illustration of another tangential flow filtration system.

Like references symbols in the various drawings indicate like elements

### DETAILED DESCRIPTION

This specification generally describes devices, systems, and methods for manufacturing an oxygen carrier (e.g., an oxygen carrier that is suitable for use in brain perfusion systems and methods). In particular, this specification describes devices, systems, and methods for isolating extracellular hemoglobin protein from the blood of worms while reducing (e.g., minimizing) protein aggregation.

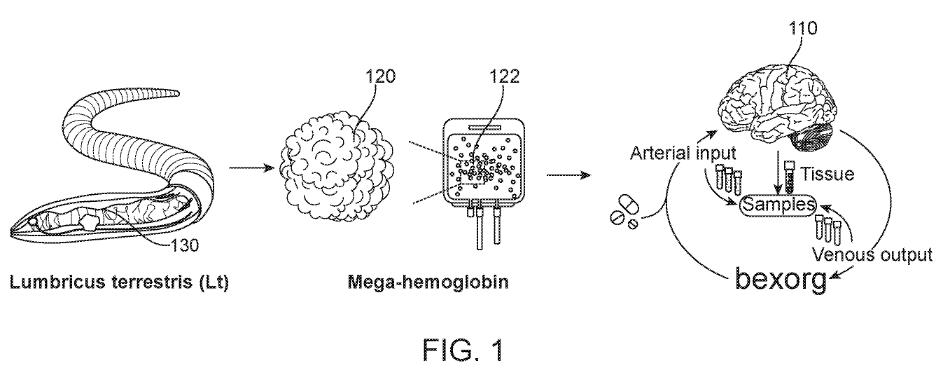

FIG. 1 is a schematic illustration of a process that includes (i) manufacturing a perfusate 122 including erythrocruorin (LtEc) 120 that has been isolated from Lumbricus terrestris (commonly known as earthworms) 130, and then (ii) using the perfusate 122 to perfuse a human brain. Perfusion of a human brain refers to the process of supplying blood or perfusate to brain’s tissues, ensuring they receive the necessary oxygen and/or nutrients for metabolic restoration.

When perfusing a brain for a 24-hour perfusion period, red blood cells are not required as oxygen levels within the brain tissue are sufficiently maintained through atmospheric oxygen. However, for perfusions of a human brain that are longer than 24 hours, the brain can become hypoxic if the perfusate does not include an oxygen carrier. In order to enable perfusions of brains that are 24 hours or longer, perfusates that include oxygen carriers can be utilized. The oxygen carriers can include substances, proteins or molecules that are configured to transport oxygen to the brain for restoring metabolic functions of an ischemic organ.

However, the blood brain barrier (BBB), which is a selective permeability barrier that protects the brain from harmful substances while allowing necessary nutrients to pass through, can present a challenge for perfusate delivery. In order to effectively deliver perfusate to the tissue of the brain, perfusates with an acellular oxygen carrier can be used. By utilizing acellular perfusate, the risk of ischemia-reperfusion injury to the perfused organ can be reduced. In addition, by utilizing acellular perfusate, perfusion processes can be scaled more easily. Advantageously, the LtEc 120 is a hemoglobin protein that is capable of passing the BBB of the brain 110.

LtEc is a high-molecular weight (about 30 nanometers and about 3.6 MDa) extracellular oxygen carrier that can serve as a blood substitute. LtEc is composed of 144 globin subunits and 36 linker chains, resulting in LtEc having 36 times more oxygen carrying capacity than human hemoglobin. Perfusates utilizing LtEc as the oxygen carrier can overcome immunogenic and blood antigen complications of certain known blood replacement therapies. Additional advantages of utilizing LtEc as the oxygen carrier in perfusate can include preventing (1) vasoconstriction, (2) disassociation, and (3) heme oxidation and hypertension, which have been observed following transfusion with other hemoglobin-based oxygen carriers. In some implementations, perfusates that contain LtEc as the oxygen carrier provide sufficient oxygen to the perfused organ (e.g., a human brain) to enable perfusion of the organ for two weeks or longer.

As schematically illustrated in FIG. 1, the LtEc 120 is isolated from earthworms 130. One challenge in LtEc isolation techniques is scaling at high volumes, which requires high purified protein yields and mitigation of potential constraints (e.g., protein aggregation). In some cases, the yield is low due to protein aggregation and protein denaturing throughout the isolation process. The technologies described in the present disclosure can reduce or prevent protein aggregation and denaturation and thereby increase yields and isolation efficacy.

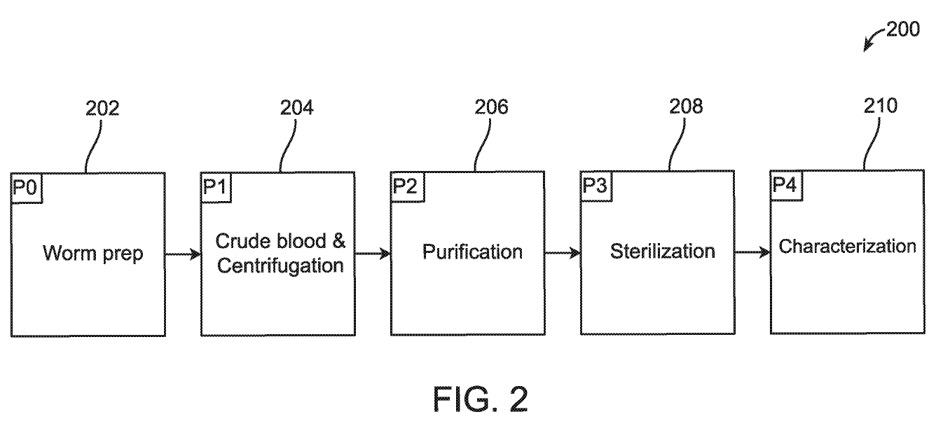

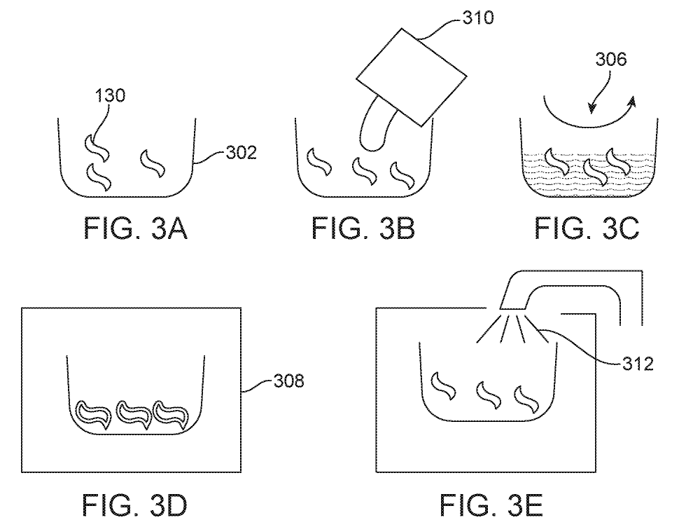

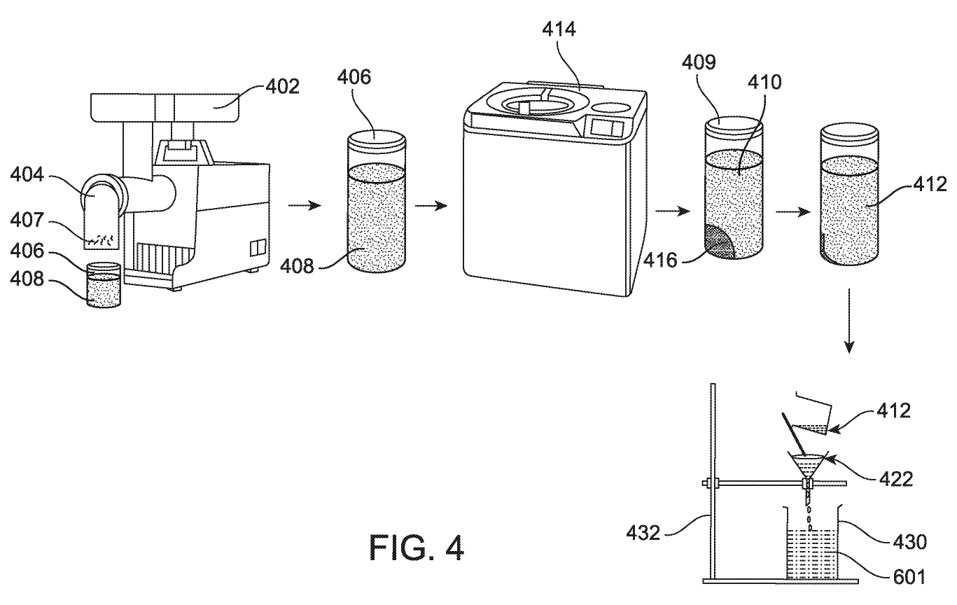

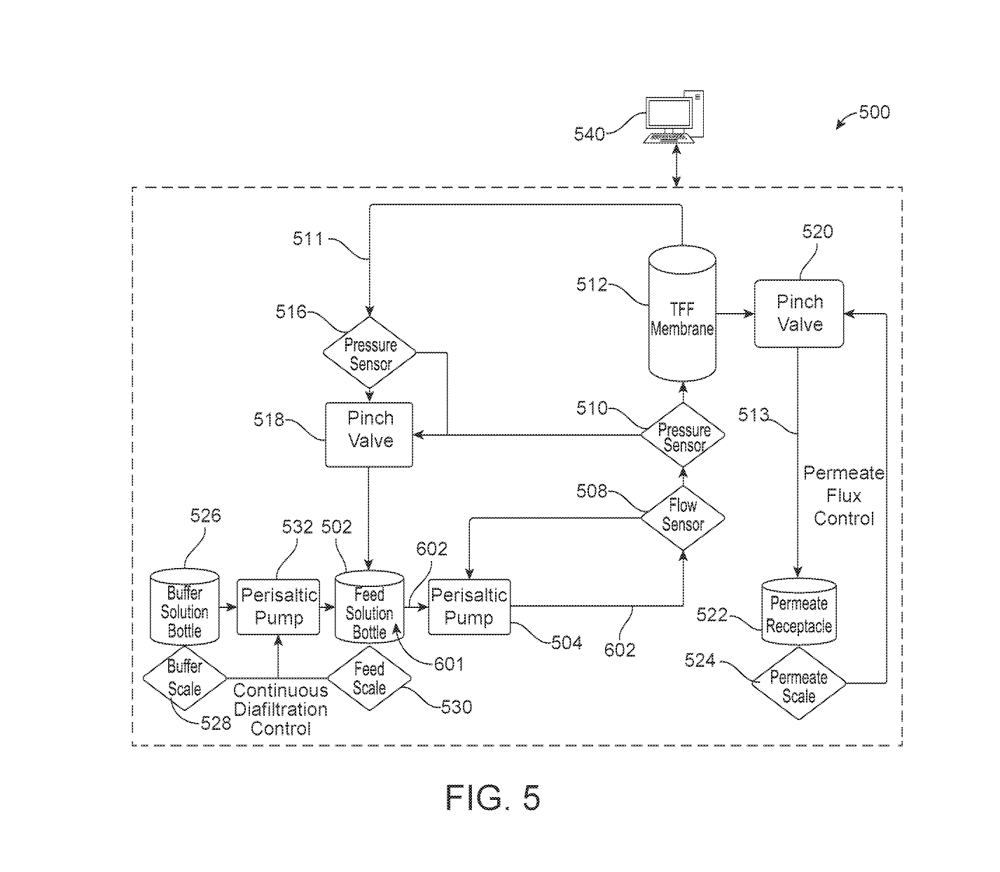

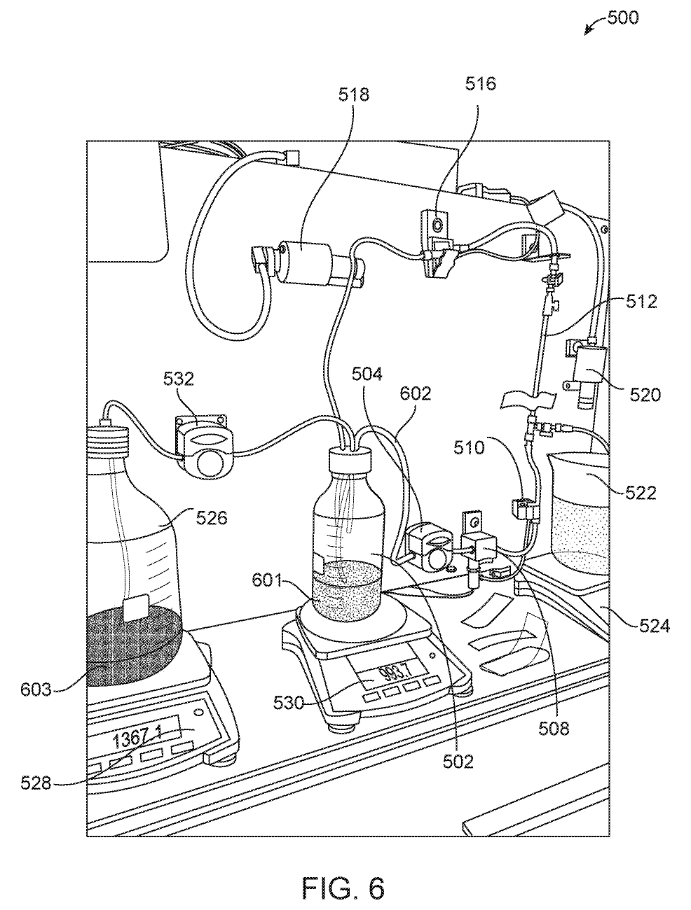

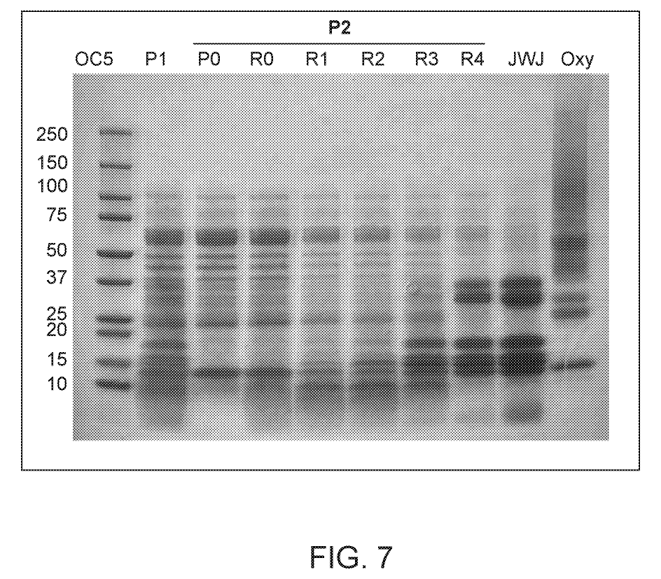

FIG. 2 is a flow diagram of an example process 200 of isolating LtEc 120 from earthworms 130, as schematically illustrated in FIG. 1. FIG. 2 provides an overview of the process, which is detailed further in the following figures: step 202 is explained in FIGS. 3A-3E, step 204 in FIG. 4, step 206 in FIGS. 5 and 6, and step 210 in FIG. 7. For ease of description, reference will be made to corresponding figures when describing each step of FIG. 2.

As shown in FIG. 2, to extract the LtEc 120 from earthworms 130, the earthworms 130 are first prepared with cornstarch to remove mucus on their skins (step 202). The crude blood of the earthworms 130 is collected and centrifuged to obtain blood supernatant (step 204). The supernatant is then purified (step 206) using a tangential flow filtration (TFF) system with continuous diafiltration control to obtain a high concentration of LtEc. The resulting concentrated blood product subsequently undergoes a sterilization process (step 208). Finally, the LtEc 120 solution can be characterized (step 210) to determine its yield, purity and/or sterility of LtEc.

At step 202, the earthworms 130 are prepped. The live earthworms 130 can be kept in a refrigerator at a temperature ranging from 0 ºC to 8 ºC (e.g., 4ºC) and mixed in their container (e.g., styrofoam container) every several days (e.g., every 3 days) to promote viability. To increase extraction yield of the LtEc from the earthworms 130, the earthworms 130 can be removed from the container and repeatedly rinsed with tap water. The earthworms 130 can be subsequently dried with a cloth to remove excess water from their skin.

The live earthworms 130 are treated with cornstarch to remove coelomic fluid (also called mucus) from their skins. The mucus can be one of the factors that cause protein aggregation during the extraction process. Without wishing to be bound by theory, it is believed that the removal of the mucus by cornstarch can sequester coelomic enzymes in the mucus that contribute to LtEc aggregation. LtEc aggregation may clog a TFF system at a later process stage when they are directed to pass through a filter membrane of the TFF system, leading to low extraction yield of LtEc. Therefore, removing mucus can help reduce protein aggregation, thereby improving the extraction yield.

FIGS. 3A-3E depict an example process for removing mucus from an exterior surface of the earthworms 130 using cornstarch. To start the process, the earthworms 130 can be placed into a container 302 (FIG. 3A). Cornstarch 310 is then applied to the earthworms (FIG. 3B), and the cornstarch 310 and earthworms 130 are thoroughly mixed 306 (FIG. 3C) such that the skins or exterior surfaces of the earthworms 130 are substantially coated with the cornstarch 310. The cornstarch 310 can be applied to the earthworms 130 by, for example, adding the cornstarch into the container 302 where the earthworms 130 are placed. The cornstarch 310 and the earthworms 130 can be mixed by hands or by a stirring rod. Typically, about 0.25 gram to about 0.5 gram of cornstarch 310 is used for each earthworm 130 to be treated. In some cases, for example, about 200 grams of cornstarch 310 is used to remove the mucus from about 500 earthworms 130.

After coating the earthworms 130 with the cornstarch 310, the earthworms 130 are incubated for a period of time in an incubator 308 (FIG. 3D) for the purpose of removal and extraction of excess cutaneous mucus from worms. In some cases, for example, the earthworms 130 are incubated for about 10 minutes to about 20 minutes (e.g., about 15 minutes). The incubator can be set to a temperature range of 20 ºC to 25 ºC (e.g., 24ºC).

The cornstarch 310 is then rinsed off of the earthworms 130 after incubation by water 312 (FIG. 3E). The rinsing can take about 15 minutes to about 30 minutes or until cornstarch is no longer visually apparent. The water 312 can be tap water.

The coating-rinsing process described above with respect to FIGS. 3A-3E can be repeated multiple times, and during each cycle the earthworms 130 can be weighed to determine their weight loss from mucosal removal. This repetitive process can be ended once a set number of repetitions is reached. In certain implementations, for example, the process is ended after 2 to 4 repetitions. Alternatively, the process can be ended when the earthworms’ weight remains substantially unchanged from one cycle to the next, indicating most of the mucus has been removed from the skins of earthworms 130. For example, the process can be ended when the weight of the earthworms 130 changes by no more than 30 grams to 50 grams.

Referring back to FIG. 2, at step 204, the crude blood of the earthworms 130 is collected and centrifuged to obtain blood supernatant. FIG. 4 depicts an example process for removing crude blood from the earthworms 130 for extracting LtEc. As shown in FIG. 4, the earthworms 130 that were prepped with cornstarch 310 and dried at step 202 are added into a grinder 402. The grinder 402 can be a standard meat grinder, including one or more cutting blades that are configured to cut earthworms 130 into small pieces when they pass through the grinder 402.

The grinder 402 cuts the earthworms 130 into small pieces. In some cases, a length of each piece of earthworms 130 is between 2mm and 5mm (e.g., 2mm). The length of each piece can be controlled by selecting the appropriate grinding plate size based on manufacture’s instruction.

These pieces of earthworms 130 (also called ground earthworms 130 in the present disclosure) then pass into a filter bag 404 that is located at the grinder outlet. The filter bag 404 can, for example, be a 50um filter bag, which can be secured to the grinder outlet by using fasteners, such as elastic bands or clips.

The filter bag 404 allows the crude blood 408 of the ground earthworms 130 to drain to a receptacle 406 while retaining the worm puree 407 in the filter bag 404. In some cases, for example, the ground earthworms 130 are drained for about 2 minutes to about 5 minutes or until it is observed that the flow of crude blood from the filter bag 404 to the receptacle 406 has stopped.

The crude blood 408 in the receptacle 406 can be further pipetted into individual falcon tubes (e.g., 50mL falcon tubes) and stored at a low temperature ranging from 0 ℃ to 8 ℃ (e.g., 4℃) until performing centrifugation by the centrifuge 414.

The individual falcon tubes with the crude blood 408 can be placed in the centrifuge 414. The crude blood 408 is purified through the centrifuge 414. The centrifuge 414 is cooled to a temperature ranging from 2℃ to 6℃ (e.g., 4℃), and the crude blood 408 that is placed in falcon tubes are added to the centrifuge 414. The crude blood 408 can be centrifuged for about 29 mins to about 31 mins (e.g., about 30 mins).

During centrifugation, the crude blood 408 is first spun at a high g-force, ranging from 9,000 g to 11,000 g (e.g., 10,000 g) for about 5 minutes to about 15 minutes (e.g.,10 minutes). The crude blood 408 can be then placed back into low temperature storage (e.g., an ice bucket, or preferably a refrigerator) for about 10 minutes to about 40 minutes. After that, the crude blood 408 is decanted into a new set of falcon tubes. This centrifugation process can be repeated once more, e.g., by spinning the crude blood 408 at 10,000g for an additional 10 minutes and decanting it into falcon tubes to create the purified end product.

After centrifugation, the individual falcon tubes can be removed from the centrifuge 414, and the crude blood 408 in the individual falcon tubes can be transferred into a receptacle 409. As shown in FIG. 4, denser components 416 in the crude blood 408 can settle at the bottom of the receptacle 409, while liquid (also called blood supernatant 410 in the present disclosure) can remain on top including LtEc. The blood supernatant 410 is collected from the receptacle 409 and diluted with buffer agents. The blood supernatant 410 can be collected from the receptacle 409 by decantation of the supernatant. The buffer agents can include tris(hydroxymethyl)aminomethane- hydrochloric acid (Tris-HCl) and/or ethylenediaminetetraacetic acid (EDTA). A dilution ratio of the blood supernatant 410 to the buffer agents can be 1:50.

The diluted solution 412 is filtered through a filter 422 (e.g., 0.22 µm filter) to remove impurities. This process may reduce air bubbles in the solution, decreasing shear stress during the TFF filtration at subsequent stages. In some examples, as shown in FIG. 4, the filtering process involves pouring the diluted solution 412 into the filter 422 that is supported by a stand 432 and collecting the filtered diluted solution 412 in a sterile bottle (e.g., a 1L: sterile bottle) 430.

The filtered diluted solution 412 can be stored in the bottle 430 at a low temperature ranging from 0℃ to 8℃ (e.g., 4℃) for extended time periods ranging from 16 hours to 48 hours to limit protein denaturation, or until the next step, e.g., diafiltration through a TFF system as described below with reference to FIGS. 5 and 6. The solution obtained from step 204 is also referred to as feed solution 601 in the present disclosure.

Referring back to FIG. 2, at step 206, the feed solution 601 from step 204 is purified using a tangential flow filtration (TFF) system. FIG. 5 illustrates a schematic illustration of the example TFF system 500. FIG. 6 is a perspective view of the example tangential flow filtration system 500 of FIG. 5. For ease of description, reference will be made to both FIGS. 5 and 6 when describing the structure of the TFF system 500. As shown in FIGS. 5 and 6, the TFF system 500 is used for isolating the LtEc 120 from the earthworms 130. In some implementations, the TFF system 500 is built and contained within a sterile environment (e.g., within a clean room).

Purification through the TFF system 500 involves two stages: concentration and diafiltration. During the first stage (concentration), in operation, the feed solution 601 obtained from step 204 can be transferred from the bottle 430 into a feed receptacle 502 that is situated on a feed scale 530 for weight measurement. The feed solution 601 is then pumped by a first peristaltic pump 504, along the fluid line 602, to a flow sensor 508 for flow rate measurement and a first pressure sensor 510 for pressure measurement. The flow sensor 508 is electrically coupled to the first peristaltic pump 504 for controlling the flow rate of the feed solution 601, and the first pressure sensor 510 is electrically coupled to a second pinch valve 518 for controlling the membrane pressure. The feed solution 601 then passes through a single TFF membrane 512 for filtration. The single TFF membrane 512 can be a 750KDa one-way filter, where molecules or impurities that are larger than 750KDa will remain in the solution, with small molecules (<=750KDa) being redirected through a first pinch valve 520 to a permeate receptacle 522. The permeate receptacle 522 can be positioned on a permeate scale 524 for weight measurement. After passing through the TFF membrane 512, the feed solution 601 flows through a second pressure sensor 516 for further pressure measurement. It then continues to the second pinch valve 518 that is used for controlling the membrane pressure based on electrical signals from the first pressure sensor 510 and/or the second pressure sensor 516. Finally, the feed solution 601 returns to the feed receptacle 502, completing one cycle of the concentration process. This cycle can be repeated multiple times, with a total duration ranging from 30 mins to 60 minutes, to obtain concentrated blood products. New batches of feed solution 601 from step 204 can be added into the feed receptacle 502 and mixed with the existing concentrated blood products. The mixed solution can then go through the TFF system 500 again for further concentration. In some implementations, the feed solution 601 is concentrated in 500mL increments until all feed solution 601 from step 204 has been added into the feed receptacle 502.

The first stage of purification through the TFF system 500 can be ended when the concentrated solution in the feed receptacle 502 reaches a predetermined volume. Alternatively, the first stage can be ended when the weight of the concentrated solution inside the feed receptacle 502 remains constant (e.g., less than 2% difference), indicating that no significant additional molecules or impurities can be filtered out. In certain implementations, for example, the predetermined volume of concentration solution ranges between 100 mL and 200 mL (e.g., 100 mL).

During this process, various sensors, including the flow sensor 508 and the pressure sensors 510, 516, are electrically coupled to a computer system 540 for maintaining a constant pressure and a constant flow rate. The pressure and flow rate can be set to levels that prevent membrane clogging while also optimizing the efficient permeate efficiency of the membrane. In some cases, the pressure is between 150 mmHg and 250 mmHg (e.g., 200 mmHg), and the flow rate is between 250 mL/min and 350 mL/min (e.g., 300 mL/min). In operation, the flow sensor 508 can send electrical signals corresponding to a flow rate measurement to the computer system 540. In response, the computer system 540 generates a control signal and transmits it to the first peristaltic pump 504 to adjust the flow rate accordingly. Similarly, the pressure sensors 510, 516 can send electrical signals corresponding to pressure measurements to the computer system 540. The computer system 540 then adjusts the second pinch valve 518 to maintain the membrane pressure.

At the second stage (diafiltration), a diafiltration process is performed. Buffer agents 603 are added to the concentrated blood product obtained from the first stage in the feed receptacle 502 to obtain a diafiltration mixture. The buffer agents 603 are stored in a buffer solution receptacle 526 and pumped into the feed receptacle 502 through a second peristaltic pump 532. The buffer solution receptacle 526 is placed on a buffer scale 528 for weight measurement.

As noted above, at the end of the first stage, the volume of the concentrated blood product is reduced to a predetermined volume (e.g., 800mL). Concentrated blood product may clog the membrane 512 due to their higher viscosity and increased risk of protein coagulation, leading to a lower filtration efficiency. Adding buffer agents 603 to the concentration blood product from the first stage can reduce the viscosity of the blood product and reduce protein coagulation, thereby improving filtration efficiency.

Buffer agents 603 can include Tris-HCl. A dilution ratio of the concentrated blood product to the buffer agents 603 can be 4:1. For example, 800mL of concentrated blood product can be diluted by 200mL of buffer agents 603, resulting in 1000mL of diafiltration mixture in total. The diafiltration mixture then goes through the TFF system 500 for further concentration. A diafiltration cycle can be completed when the volume of diafiltration mixture in the feed receptacle 502 is lowered to a predetermined volume between 500 mL and 800 mL (e.g., 800 mL).

The second stage can involve multiple diafiltration cycles ranging from 10 cycles to 40 cycles in total (e.g., 11 diafiltration cycles). After each diafiltration cycle, the volume of the diafiltration mixture is lowered to the predetermined volume, and additional buffer agents 603 is added to the feed receptacle 502 through the second peristaltic pump 532. In some implementations, starting from the second diafiltration cycle, a ratio of the reconcentrated blood product (e.g., the remaining diafiltration mixture after a diafiltration cycle) to the buffer agents 603 is 9 to 1. For example, at the end of a previous cycle, the volume of the diafiltration mixture is lowered from 1000mL to 900mL. An additional 100 mL buffer agents 603 is then added into the reconcentrated blood product, restoring the total volume to 1000mL. This ensures that the diafiltration mixture begins the next diafiltration cycle at a consistent volume of 1000mL, maintaining a constant flow volume in the TFF system 500.

A continuous diafiltration process can be implemented by automating the addition of the buffer agents 603 into the reconcentrated blood product before each diafiltration cycle. For example, in operation, the feed scale 530 monitors the weight of the diafiltration mixture in the feed receptacle 502 during each diafiltration cycle. When a predetermined weight (corresponding to a predetermined volume, e.g., 800mL for the first diafiltration cycle, 900mL for the subsequent diafiltration cycles) is reached, the feed scale 530 sends a signal to the computer system 540. The computer system 540 then transmits a control signal to the second peristaltic pump 532 to pump a specific amount (e.g., 200mL for the first diafiltration cycle, 100mL for the subsequent diafiltration cycles) of buffer agent 603 into the feed receptacle 502. The precise amount of pumped buffer agents 603 can be measured by the buffer scale 528, which is also coupled to the computer system 540. This automated system enables a continuous diafiltration process with uninterrupted transitions between each diafiltration cycle and helps maintain a constant flow within the TFF system 500. In contrast, a discontinuous diafiltration process (without using the technologies described in the present disclosure) may require temporarily removing the feed receptacle 502 from the TFF system 500 for adding buffer agents 603. Therefore, the continuous diafiltration approach described in the present disclosure can reduce labor costs and increase filtration efficiency within a given time frame. In some cases, the filtration efficiency of the continuous diafiltration process is improved fourfold compared to a discontinuous diafiltration process.

During the diafiltration process, a small sample (e.g., 1 mL) of reconcentrated blood product and permeate solution (e.g., the solution that is filtered out by the TFF membrane 512 and stored in the permeate receptacle 522) can be obtained to assess purity of the sample. A 1mL sample of reconcentrated blood can be collected from an apical circuit stopcock using a syringe, while 1mL sample of permeate solution can be drawn from a waste line stopcock suing a syringe. The apical circuit stopcock can be coupled to the retentate fluid line 511, while the waste line stopcock can be coupled to the permeate fluid line 513. The sample purity can be assessed using one or more of Drabkin’s assay, gel electrophoresis, mass spectroscopy, dynamic light scattering, analytical high-performance liquid chromatography (HPLC), spectrometry, the Limulus amebocyte lysate (LAL) assay. After completing the last diafiltration cycle (e.g., the eleventh diafiltration cycle), the blood product can be reconcentrated to a final volume ranging from 100 mL to 200 mL (e.g., 100mL). In some implementations, the purification process performed using the TFF system 500 can generate 30 grams or more of isolated LtEc.

Referring back to FIG. 2, after the purification process by the TFF system 500 (step 206 of FIG. 2), the reconcentrated blood product can undergo a sterilization process (step 208 of FIG. 2). This sterilization process includes passing the reconcentrated blood product through a filter (e.g., 0.45 uM) to remove any remaining impurities and generate a final blood product. After sterilization, the final blood product can be frozen in a low temperature ranging from -78 ℃ to -81℃ (e.g., -80℃) before a characterization is performed.

At step 210, a characterization is performed to assess LtEc’s purity. FIG. 7 illustrates an example gel characterization of the LtEc in the final blood product obtained from step 208. Gel characterization can utilize Drabkin’s assay and gel electrophoresis. The Drabkin's assay is to measure hemoglobin in a blood sample by converting it to cyanmethemoglobin, a stable pigment with a maximum absorption at 540 nanometers (nm). Gel electrophoresis is a laboratory technique that separates molecules by size and charge using an electric field and a porous gel.

In FIG. 7, y axis represents different protein bands corresponding to different types of proteins, while x axis represents characterization results at different steps during an isolating process depicted in FIG. 2. For example, P1 represents a characterization made after centrifugation (step 204 of FIG. 2), and P2 represents characterization made at different cycles in the TFF process (step 206 of FIG. 2). JWJ column in FIG. 7 represents a characterized band distribution of the final blood product obtained from step 208 of FIG. 2. Based on the band distribution, purity of LtEc in the final blood product can be determined through visual inspection of the gel electrophoresis band. In the example shown in FIG. 7, 6.8g of pure LtEc is obtained from 500 earthworms 130. After characterization, the final blood product can be frozen under a low temperature ranging from -78℃ to -81℃ (e.g., -80℃) for long term storage.

The purity of LtEc isolated using the systems and methods described herein can also be characterized using one or more of mass spectroscopy, dynamic light scattering, analytical high-performance liquid chromatography (HPLC), spectrometry, and the Limulus amebocyte lysate (LAL) assay. The quality control metrics generated using one or more of these testing methods can be measured after each perfusion process performed using perfusate containing the isolated LtEc. In some implementations, the systems and methods for isolating LtEc described herein provide LtEc that is sufficiently pure such that the coefficient of variation for quality control tests performed during perfusion processes utilizing the isolated LtEc is less than 10%.

In some implementations, the purity of the isolated LtEc is tested to confirm that the isolated LtEc is clinical grade, low-endotoxin LtEc. For example, the isolated LtEc can be tested using analytical HPLC and dynamic light scattering to confirm that the isolated LtEc compound is pure, soluble LtEc. In some implementations, the isolated LtEc is tested using a Limulus amebocyte lysate assay to confirm that the isolated LtEc contains less than 0.1 endotoxin units (EU) per milliliter (e.g., < 0.1 EU/mL).

In some implementations, the oxygen carrying capacity of the isolated LtEc can be tested using one or more assays including, but not limited to, spectrophotometry.

In some implementations, the isolated LtEc can be tested to confirm that the isolated LtEc achieves stable metabolic outputs. For example, during perfusion of a brain using perfusate containing the isolated LtEc, the rates at which the brain (1) consumes oxygen and glucose and (2) produces CO₂ and lactate can be measured and compared to physiological rates of oxygen and glucose consumption and CO₂ and lactate production to determine whether the perfusate containing the isolated LtEc results in stable, physiological metabolic outputs.

In some implementations, the isolated LtEc can be tested to confirm that the isolated LtEc provides an adequate supply of oxygen to tissue (e.g., achieves normoxia) during perfusion of a brain. For example, the level of hypoxia of a brain being perfused using perfusate containing the isolated LtEc can be determined by analyzing a hypoxia gene signature in a variety of brain regions, including hypoxia-inducible factor-1a, HIF1α, and its targets. The hypoxia gene signature measured for the perfused brain can be compared to known population variability of hypoxia gene signatures in living human subjects to determine whether normoxia is achieved during perfusion utilizing the isolated LtEc.

The analyses of whether the isolated LtEc achieves stable metabolic outputs and provides an adequate supply of oxygen to tissue (e.g., achieves normoxia) could be performed across multiple brain perfusions, each utilizing a different concentration of LtEc across a range of concentrations, in order to identify an optimal concentration of LtEc for brain perfusion perfusate and demonstrate the biological effectiveness of the selected LtEc concentration.

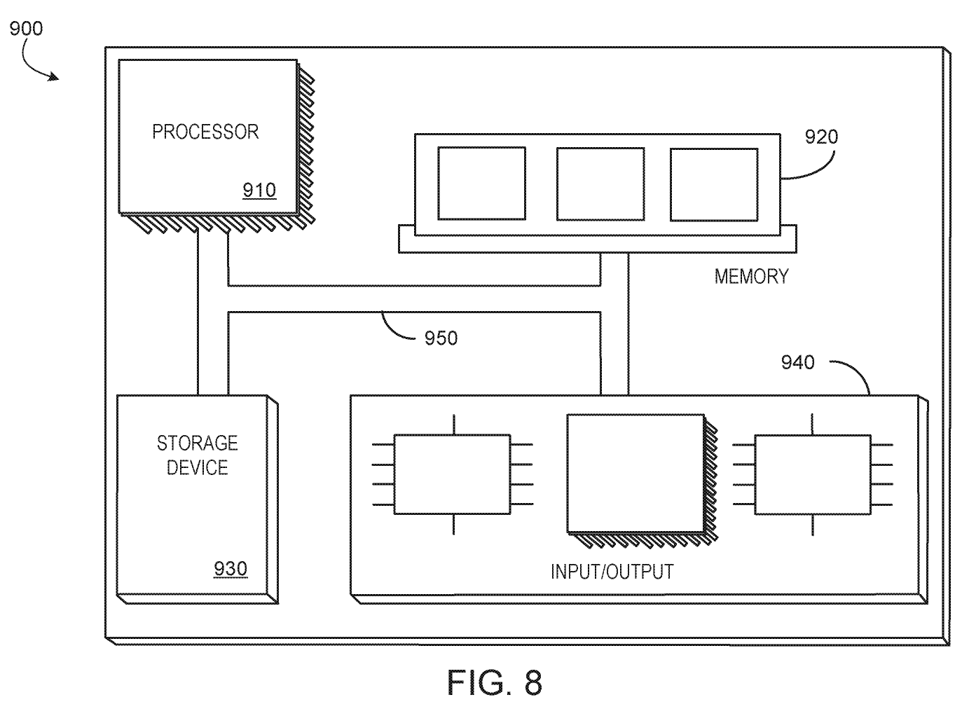

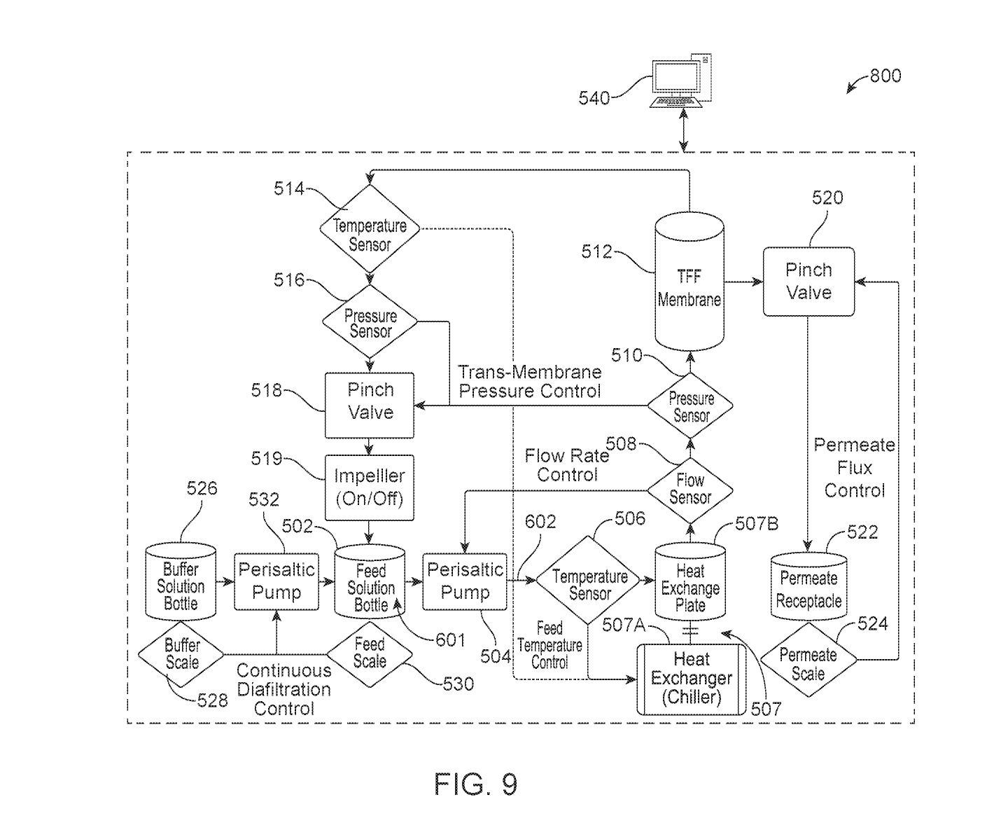

FIG. 8 is a block diagram of an example computer system 900 that can be used in the TFF system 500 described herein. For example, referring to FIG. 5, the computer system 540 could be an example of the system 900 described here. The system 900 includes a processor 910, a memory 920, a storage device 930, and an input/output interface 940. Each of the components 910, 920, 930, and 940 can be interconnected, for example, using a system bus 950. The processor 910 is capable of processing instructions for execution within the system 900. The processor 910 can be a single-threaded processor, a multi-threaded processor, or a quantum computer. The processor 910 is capable of processing instructions stored in the memory 920 or on the storage device 930. The processor 910 may execute operations such as receiving signals from one or more sensors (e.g., the pressure sensors 510, 516 of FIGS. 5, 6 and 9, the flow sensor 508 of FIGS. 5, 6 and 9, and the temperature sensors 506, 514 of FIG. 9) and controlling or more elements of the system (e.g., the first peristaltic pump 504 of FIGS. 5, 6 and 9, the second peristaltic pump 532 of FIGS. 5, 6 and 9, the heat exchange system 507 of FIG. 9, the second pinch valve 518 of FIGS. 5, 6 and 9) based on the received signals.

The memory 920 stores information within the system 900. In some implementations, the memory 920 is a computer-readable medium. The memory 920 can, for example, be a volatile memory unit or a non-volatile memory unit. In some implementations, the memory 920 stores a data structure. In some implementations, multiple data structures are used.

The storage device 930 is capable of providing mass storage for the system 900. In some implementations, the storage device 930 is a non-transitory computer-readable medium. The storage device 930 can include, for example, a hard disk device, an optical disk device, a solid-date drive, a flash drive, magnetic tape, or some other large capacity storage device. The storage device 930 may alternatively be a cloud storage device, e.g., a logical storage device including multiple physical storage devices distributed on a network and accessed using a network.

The input/output interface 940 provides input/output operations for the system 900. In some implementations, the input/output interface 940 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). In some implementations, the input/output device includes driver devices configured to receive input data and send output data to other input/output devices, e.g., keyboard, printer and display devices. In some implementations, mobile computing devices, mobile communication devices, and other devices are used.

In some implementations, the system 900 is a microcontroller. A microcontroller is a device that contains multiple elements of a computer system in a single electronics package. For example, the single electronics package could contain the processor 910, the memory 920, the storage device 930, and input/output interfaces 940.

Although an example processing system has been described in FIG. 8, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

A number of embodiments have been described. Nevertheless, it will be understood that various modifications may be made without departing from the spirit and scope of the disclosure.

The process described above in reference FIGS. 1-8 involve the use of earthworms 130 for isolating LtEc. In some implementations, the earthworms 130 are Canadian Night Crawlers.

While the mucus removal process illustrated in FIGS. 3A-3E involves the use of cornstarch 310 as a drying agent, other drying agents may alternatively be used. In some implementations, drying agents include flour, cornmeal, etc.

While the process described above in reference FIG. 4 involves the use of a grinder 402, in some implementations, a grinder 402 is not used. Instead, the crude blood is removed from the earthworms 130 by manual cutting using multi-blade scissors. During this process, each earthworm 130 obtained from step 202 of FIG. 2 can be sliced manually with the multi-blade scissors into multiple small pieces, with each piece being approximately 2 mm in length. Ground earthworms 130 are then placed in the filter bag 404, allowing the crude blood 408 to drain into the receptacle 406. In some implementations, a cutting with multi-blade scissors can be employed for processing less than 500 earthworms 130, while the grinder 402 can be used for processing more than 500 earthworms 130.

While the process described above in reference to FIG. 4 involves storing the crude blood 408 filtered from the filter bag 404 at a low temperature ranging from 0 ℃ to 10 ℃ (e.g., 4℃) until performing centrifugation by the centrifuge 414, in some cases, this storage process is not required. The crude blood can directly undergo centrifugation process after it is collected.

While the process described above in reference to FIG. 4 involves a manual filtration of the diluted solution 412 through the filter 422, other filtration methods may alternatively be used. In some implementations, the bottles of diluted solution 412 can be attached to a Master Flex L/S Easy Build pump and filtered via a 0.22um Cytiva Whatman™ Polycap Disposable Capsules, 36 AS from Fisher Scientific. The pump can be set to a speed of ranging between 70 and 150 mL/min (e.g., 70 mL/min), and the diluted solution 412 can be drained into a sterile bottle 430.

While the process described above in reference to FIG. 4 involves storing the filtered solution in the 4℃ refrigerator in preparation of the subsequent filtration stage, in some implementations, this storage process is not required. The filtered solution can directly proceed to the TFF system for purification.

The TFF system 500 have been described with respect to the processes illustrated in FIGS. 5 and 6. In some implementations, as illustrated in FIG. 9, a TFF system 800 additionally includes a first temperature sensor 506, a second temperature sensor 514, and a heat exchange system 507. In operation, the feed solution 601 is pumped to the first temperature sensor 506 and the heat exchange system 507. The first temperature sensor 506 and the heat exchange system 507 can be configured to keep a stable temperature between 3 ℃ and 10 ℃ (e.g., 10℃) of the feed solution 601. A constant temperature (e.g., within a 5% variance) can keep the structural integrity of LtEc and reduce shear stress in LtEc. The feed solution 601 is then directed to the TFF membrane 512 for filtration. From there, the feed solution 601 flows through the second temperature sensor 514 for another temperature measurements. Both the first temperature sensor 506 and the second temperature sensor 514 can be coupled to the heat exchange system 507 through the computer system 540. The heat exchange system 507 can include a chiller 507A for decreasing the temperature and a heater 507B for increasing the temperature. Based on the temperature sensor signals or control signals from the computer system 540, the heat exchange system 507 can operate the chiller 507A and/or the heater 507B to maintain a constant temperature (e.g., 10℃).

In addition, the TFF system 800 includes an impeller 519, which is configured to mix the solution inside the feed receptacle 502 thoroughly to improve uniformity. For example, the impeller 519 can operate when additional feed solution 601 is introduced during the first stage of TFF process (concentration), or when buffer agents 603 are added in the second stage (diafiltration).

While the TFF processes described above in reference to FIGS. 5, 6 and 9 involve the use of two pressure sensors 510, 516, in some implementations, a single pressure sensor is used, either upstream of the TFF membrane 512 or downstream of the TFF membrane 512.

While the TFF processes described above in reference to FIG. 9 involves the use of two temperature sensors 506, 514, in some implementations, a single temperature sensor is used, either upstream of the TFF membrane 512 or downstream of the TFF membrane 512.

While the TFF processes described above in reference to FIGS. 5, 6 and 9 involve the use of the computer system 540 as an intermediary for communications between different components, these components can directly communicate with each other. For example, the flow sensor 508 can be configured to directly send measurement signals to the first peristaltic pump 504, which can then receive and act upon these signals. Similarly, the first pressure sensor 510 and the second pressure sensor 516 can be configured to send signals to the second pinch valve 518, which can receive and respond to these signals. Additionally, the temperature sensors 506, 514 and the heat exchange system 507 can communicate directly with each other.

While the process described above in reference to FIGS. 5 and 6 involves storing the final blood product in a low temperature ranging from -79℃ to -81℃ (e.g., -80℃) before a characterization process is performed, in some implementations, this storage process is not required. The final blood product can directly proceed to the characterization step in the extraction process (e.g., step 210 of FIG. 2).

While the characterization process described above in reference to FIG. 7 involves Drabkin’s assay and gel electrophoresis, other characterization methods can be utilized, for example, Evelyn/Malloy method.

While the extracted LtEc has been described to be frozen at a low temperature (e.g., -80℃) for long term storage after characterization, in some implementations, lyophilization is used instead for long term storage.

While the perfusate has been described as being used to perfuse human brains, the perfusate can alternatively or additionally be used to perfuse any of various other mammalian brains, including monkey brains, porcine brains, etc., as well as non-mammalian brains, including mice brains, rats brains, etc. Further, while the perfusate has been described as being used to perfuse brains, the perfusate can be used to perfuse any of various other organs, including liver, heart, pancreas, kidney, lung, etc.

While the LtEc has been described to be utilized for perfusing organs, the LtEc may be used for purposes other than perfusion, including stroke recovery, trauma care, wound healing, myocardial infarction, ischaemic damage recovery, organ resuscitation and recovery, and/or enhancing cell cultures, organoids, organs-on-chip, and enzymatic systems used for assay development.

While the processes described above in reference to FIGS. 1-8 involve the use of LtEc, other hemoglobin proteins may alternatively be used. Examples of other such hemoglobin proteins include other hemoglobin-based oxygen carriers derived from annelid species.

A number of embodiments have been described. Nevertheless, it will be understood that various modifications may be made without departing from the spirit and scope of the disclosure.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method for isolating extracellular hemoglobin protein from worms, the method comprising:
removing coelomic fluid from an exterior surface of the worms using a drying agent;
removing blood from the worms;
performing centrifugation on the blood to generate a blood supernatant;
concentrating, by a tangential flow filtration system with a single membrane, the blood supernatant to form a concentrated blood product; and
performing, by the tangential flow filtration system, diafiltration on a diafiltration mixture comprising the concentrated blood product to obtain a final blood product.

2. The method of claim 1, wherein the drying agent comprises cornstarch, and removing the coelomic fluid from the exterior surface of the worms comprises coating an outer surface of the worms with the cornstarch.

3. The method of claim 1, wherein removing the blood from the worms comprises grinding the worms by a grinder to obtain ground worms and draining the blood from the ground worms.

4. The method of claim 3, wherein draining the blood from the ground worms comprises placing the ground worms in a filter bag and flowing the blood through the filter bag into a receptacle.

5. The method of claim 1, further comprising, before concentrating the blood supernatant by the tangential flow filtration system, filtering the blood supernatant.

6. The method of claim 1, wherein concentrating the blood supernatant to form the concentrated blood product comprises:
flowing the blood supernatant from a feed receptacle through the single membrane to generate a concentrated blood solution, wherein the single membrane comprises a 750 kDa one-way filter; and
flowing the concentrated blood solution back to the feed receptacle.

7. The method of claim 1, further comprising adding a buffer solution to the concentrated blood product to obtain the diafiltration mixture.

8. The method of claim 7, wherein the buffer solution is configured to be automatically added to a feed receptacle of the tangential flow filtration system throughout the diafiltration on the diafiltration mixture to maintain a constant volume of the diafiltration mixture within the tangential flow filtration system.

9. The method of claim 1, wherein performing the diafiltration on the diafiltration mixture comprises controlling a temperature of the diafiltration mixture throughout the diafiltration based on one or more signals received from a temperature sensor.

10. The method of claim 1, wherein performing the diafiltration on the diafiltration mixture comprises controlling a flow rate of the diafiltration mixture based on one or more signals received from a flow sensor.

11. The method of claim 1, wherein performing the diafiltration on the diafiltration mixture comprises controlling a pressure of the diafiltration mixture passing through the single membrane based on one or more signals received from at least one pressure sensor.

12. The method of claim 1, wherein the extracellular hemoglobin protein is erythrocruorin.

13. The method of claim 1, wherein the worms comprise Lumbricus terrestris earthworms.

14. A system for isolating extracellular hemoglobin protein from worms, the system comprising:
a device for removing blood from the worms;
a centrifuge configured to perform centrifugation on the blood to generate a blood supernatant; and
a tangential flow filtration system with a single membrane, the tangential flow filtration system configured to concentrate the blood supernatant to form a concentrated blood product and to perform diafiltration on a diafiltration mixture comprising the concentrated blood product to isolate the extracellular hemoglobin protein from the blood.

15. The system of claim 14, wherein the device for removing blood from the worms comprises a meat grinder.

16. The system of claim 14, wherein the tangential flow filtration system comprises:
a feed receptacle;
a fluid line fluidly coupling the feed receptacle to the single membrane;
a peristaltic pump configured to flow the diafiltration mixture from the feed receptacle, along the fluid line, and through the single membrane; and
at least one flow sensor fluidly coupled to the fluid line downstream of the single membrane, wherein the peristaltic pump is configured to be controlled based at least in part on one or more signals generated by the at least one flow sensor.

17. The system of claim 14, wherein the tangential flow filtration system comprises:
a feed receptacle;
a fluid line fluidly coupling the feed receptacle to the single membrane;
a pinch valve coupled to the fluid line downstream of the single membrane; and
at least one pressure sensor fluidly coupled to the fluid line, wherein the pinch valve is configured to be controlled based at least in part on one or more signals generated by the at least one pressure sensor.

18. The system of claim 14, wherein the tangential flow filtration system comprises:
at least one temperature sensor; and
a heat exchanger system, wherein the heat exchanger system is configured to control a temperature of the diafiltration mixture based on one or more signals received from the at least one temperature sensor.

19. The system of claim 14, wherein the extracellular hemoglobin protein is erythrocruorin.

20. The system of claim 14, wherein the worms comprise Lumbricus terrestris earthworms.
